(19) United States
(12) Patent Application Publication
(10) Pub. No.: US 2008/0197048 A1
(43) Pub. Date: Aug. 21, 2008

(54) Use of bone marrow stromal cells for the treatment of tendons and/or ligaments damages

(75) Inventors: Antonio Crovace, Valenzano (Bari) (IT); Edda Francioso, Valenzano (Bari) (IT)

Correspondence Address: NIXON & VANDERHYE, PC, 901 NORTH GLEBE ROAD, 11TH FLOOR, ARLINGTON, VA, 22203, US

(73) Assignee: UNIVERSITA' DEGLI STUDI DI BARI, Bari (IT)

(21) Appl. No.: 12/078,062

(22) Filed: Mar. 26, 2008

Related U.S. Application Data
(63) Continuation-in-part of application No. PCT/IT2005/000549, filed on Sep. 26, 2005.

(51) Int. Cl. A61K 35/32; A61M 5/178

(52) U.S. Cl. 206/571; 424/93.7

(57) ABSTRACT

Object of the present invention is the use of bone marrow stromal cells, in particular autologous stromal cells, for the treatment of injuries to tendons and/or ligaments in mammals, in particular horses and especially racehorses. A further object of the present invention is a process for the treatment of injuries to tendons and/or ligaments in mammals that includes the delivery of bone marrow stromal cells, which are preferably autologous, into the lesions and a composition of bone marrow stromal cells, which are preferably autologous, for the treatment of the injured tendons and/or ligaments of a mammal, in particular a horse.

### CROSS-REFERENCE TO RELATED APPLICATION

This application is a Continuation-in-Part of International Application No. PCT/IT2005/000549, filed 26 Sep. 2005, which designated the U.S. The entire contents of that application is hereby incorporated by reference.

### STATE OF THE ART

Flexor tendon injuries are one of the main causes for the suspension of sporting activity in both humans and mammals, particularly in the case of racehorses. Indeed, the treatment of injuries using traditional techniques does not allow full recovery and return to normal levels of sporting performance. Furthermore, sometimes apparently normal recoveries are followed by relapses. Most commonly used treatments have provided marginal results which are always proportionate to the seriousness of the initial injury. Indeed, such treatments do not allow the tendon tissue to be regenerated, which is in any case marked by a scar, which depends on the extent of the initial injury and the type of therapy used. Some of the aforementioned treatments consist of, for example, hyaluronic acid injections, iodine injections, acupuncture, thermo-cauterization, cryo-cauterization, splitting, rest and rehabilitation. However, as is well known, the scar tissue, which is in itself anelastic, does not function in the same way as the tendon tissue and this is why it is unlikely that sporting performance will return to initial levels.

Recently in this field, for the reasons described above, there has also been considerable interest in the potential therapeutic effects of tissue regeneration.

For example, some therapies for treating flexor tendon injuries in racehorses have been suggested which involve the use of growth factors; an example is the therapy reported in WO 200182951, which describes the use in mammals of the Insulin-like Growth Factor (IGF) to treat tendon or ligament injuries which are strain-induced, caused by wear or physical force, deriving from pathological conditions or provoked by other causes.

Also well known is the treatment of the aforementioned injuries with the use of bone marrow in toto, with the purpose of providing a certain amount of cells with development potentials and growth factors. In this case, the use of bone marrow in toto means that, in addition to the stem cells that are actually useful for repairing the tendon or the ligament, at the same time other components of the bone marrow are also delivered to the damaged tendon site; these components may be fat cells, for example, which are present in a greater quantity that the quantity of stem cells that are needed, and which may be extremely damaging. Furthermore, the quantity of stromal cells present in the bone marrow is rather low, and in order for a sufficient minimum amount to be administered it is necessary to treat the injury with large quantities, and therefore large volumes, of bone marrow; this implies that much higher quantities are administered of various components other than the useful component represented by the stromal cells.

Research has also been carried out into the use of expanded Bone Marrow Stromal Cells (BMSC), which have demonstrated a potential therapeutic effect. Stromal cells are pluripotent, indifferentiated stem cells which are found inside bone marrow and which are able to differentiate themselves in numerous tissues of stromal origin, in the presence of a favourable microenvironment. However, the use of stromal cells according to prior art involves isolating and selecting said cells, and also incubating them with a differentiating agent for a sufficient length of time in order to modify the cell phenotype, before they can be used to treat the injury.

Recent studies have brought to light various techniques for treating injuries to tendons or ligaments in racehorses, for example, which involve the in vitro isolation, characterisation and expansion of equine Mesenchymal Stem Cells (MSC) (when treating racehorses). This technique allow to re-implant a large number of autologous MSC cells which are able to differentiate into tenocytes and regenerate the tendon matrix following the injury, in the horse's damaged flexor tendon. In this case, the composition of mesenchymal stem cells must first be enriched, for example through fractioning and concentration, compared to the natural source of said cells or the tenocytes that derive from them.

It is clear that treatments that use stem cells as described above involve the necessity to treat the aforesaid cells in vitro before they can be used, to allow them to be enriched, expanded and if necessary also differentiated. This involves a long period needed to prepare the cell composition being used, more laborious and complex procedures for preparing said composition and, as a result, very high costs.

### OBJECTS OF THE INVENTION

The object of the present invention is to provide a cell composition for the treatment of injuries to tendons and/or ligaments in mammals, in particular horses and especially racehorses, which can be used directly without undergoing in vitro treatments such as enrichment, expansion, differentiation.

A further object of the present invention is to make available a cell composition for the treatment of injuries to tendons and/or ligaments in mammals, in particular horses and especially racehorses, which contains practically no other components of the bone marrow that may be highly dangerous.

A further object of the present invention is to make available a cell composition for the treatment of injuries to tendons and/or ligaments in mammals, in particular horses and especially racehorses, which enables direct intervention on the injury, which avoids the formation of scar tissue, which leads to total recovery within a short period of time and reduces the likelihood of relapses to a minimum.

A further object of the present invention is to provide a cell composition for the treatment of injuries to tendons and/or ligaments in mammals, in particular horses and especially racehorses, which allows the composition to be delivered to the injection site and does not lead to any inflammatory reaction, or any rejection symptoms.

### DESCRIPTION OF THE INVENTION

These and other objects and corresponding advantages, which shall be explained in greater detail in the description below, are achieved by fresh bone marrow mononuclear stromal cells (BMSC) for the treatment of injuries to tendons and/or ligaments in mammals.

According to the present invention, the term “fresh” when referring to said bone marrow mononuclear stromal cells (BMSC) implies that said cells have not undergone any in vitro treatment for enrichment and/or expansion before they are used to repair the lesion, nor have they undergone only in vitro differentiation. Furthermore, also according to the present invention, said bone marrow mononuclear stromal cells (BMSC) are autologous cells.

Also object of the invention is the use of fresh bone marrow mononuclear stromal cells (BMSC), specifically autologous ones, for the preparation of a medicament for the treatment of injuries to tendons and/or ligaments in mammals.

Always according to this invention, said mammals are humans, horses, dogs, camels, sheep, and particularly racehorses. Said injuries to tendons and/or ligaments can be strain induced, as often happens in the case of competition or racing mammals, in particular racehorses, but may also be induced by damage accumulated over a long period of time.

Another object of this invention is a process for the treatment of injuries to tendons and/or ligaments in a mammal which involves the use of fresh bone marrow mononuclear stromal cells, said process involving the delivery of a suitable quantity of said fresh cells directly to the point of the injury.

Therefore, as described above, fresh bone marrow mononuclear stromal cells are used to great advantage for the therapy and treatment in particular of tendon injuries in horses, without the use of techniques to expand said cells, nor growth factors. In this way, the time needed for operating is reduced considerably, since the cells do not have to undergo in vitro expansion, growth or differentiation treatments before they can be used; as a result, the technique is much more simple and considerably less demanding, also from an economical point of view.

In summary, the process according to the invention involves the isolation of fresh autologous bone marrow mononuclear stromal cells which, once suitably separated and prepared, are subsequently delivered under ultrasound guidance into the injury, which may be a tendon injury, leading to healing which may be detected by ultrasound as soon as 12 weeks later.

Merely by way of a non-limitative example of the present invention, below a description is given of some experimental phases of the process according to the invention, which include the technique used for diagnosing the injury, the isolation and implantation of fresh BMSC cells and the ultrasound and clinical results obtained in the treatment of several tendon injuries in a horse.

The study included horses that are regularly trained for sporting activity. The animals underwent a clinical examination and an ultrasound examination, which confirmed the suspected diagnosis. The clinical examination showed swelling in the region of the flexors, and showed that the tendons affected by the injury were painful to the touch. When walking, the subjects demonstrated lameness to a degree of 3/5. The ultrasound scan was carried out using a General Electrics, Logic Alfa100 ultra-sonograph and a 7.5 MHz linear probe. When assessing the injuries, the following factors were taken into account: the percentage ratio between the “Cross Sectional Area (CSA) of the injury in relation to the total CSA of the tendon” and the “Fiber Pattern Score” (a score given according to the degree of disintegration of the fibrils in a longitudinal scan of the tendon) and the “Echo Score” (a score given to the injury based on the echogenicity of the injury in a transversal scan of the tendon). The location of the injury and its ultrasonographic characteristics are given in Table 1 below:

| | | | | | | | | % CSA |
|---|---|---|---|---|---|---|---|---|
| | | | | | Time since | ECHO | | INJURY to |
| PATIENT | AGE | LIMB | TEND/LEG | ZONE | injury | SCORE | FPS | MIZ |
| 001 | 3 years | FR | SDFT | 2b/3a | 2 weeks | −2 | 3 | 100 |
| 002/a | 2 years | FR | ICL | 1a | 1 week | −3 | 3 | 18.70 |
| 002/b | | FR | SDFT | 3a | 1 week | −2 | 3 | 14.60 |
| 003 | 13 years | FR | SL (lat | 2b | 4 weeks | −2 | 3 | 34.7 |
| | | | branch) | | | | | |
| 004 | 5 years | HR | SDFT | 2b | 2 weeks | −3 | 3 | 28 |
| 005 | 5 years | FL | SDFT | 2b | 2 weeks | −3 | 3 | 27.8 |
| 006 | 2 years | FL | SDFT | 3a/3b | 3 weeks | −3 | 3 | 54.10 |

Once it has been decided to carry out cell treatment, bone marrow was extracted from the wing of the ilium, with the animal standing in a crush, under pharmacological sedation with detomidine (0.02 mg/kg) and butorphanol (0.02 mg/kg). The site of extraction was anaesthetised with the introduction of 20 ml of 2% lidocaine around the wing of the ilium. The skin at the site of extraction was surgically prepared by means of trichotomy and the surgical preparation of the site. To carry out the extraction, 8 Gauge JAMSHIDI bone marrow biopsy needles were used, which were inserted at the level of the tuberosity of the ilium to a depth of around 6 cm. From this site a variable quantity of between 40 and 80 ml of bone marrow was extracted with 50 ml sterile syringes that had previously been flushed with heparin. The extracted bone marrow was transferred into Falcon test tubes containing 0.6-0.7 ml/20 ml of heparin.

Subsequently, the samples were diluted in PBS (phosphate buffered saline, pH 7.2) with a dilution of 1:2.

The mononucleated cells (MNC) were coloured with a nuclear colouring agent (0.1% of Methyl Violet in 0.1 M of citric acid), 200 μl of bone marrow diluted in PBS 1:10 was mixed with 200 μl of colouring, after approximately 10-15 minutes the cell count was determined using a Burker chamber.

The diluted bone marrow was stratified, at a ratio of 1:1, in Biocoll separating solution (Ficoll) with a density of 1077 and was centrifuged at 2000 rpm for 30 minutes.

The centrifugation allowed the separation of the portion containing the cells, which were visible due to the formation of an opalescent ring between the two phases; the portion was recovered and centrifuged again at 2000 rpm for 10 minutes.

The pellet obtained was washed with PBS and physiological solution 2 to 3 times, centrifuged at 2000 rpm for 10 minutes and, having reduced it to 10 ml, a new cell count was carried out in the Burker chamber.

Having defrosted 2 ml of fibrin glue (Tissucol), a dilution with PBS was carried out in a 1:2 ratio, said dilution was added to the cell pellet.

Lastly, the cells obtained were distributed all over the surface of the biomaterial, to which thrombin was added in order to obtain the formation of a clot.

Two ml of fibrin glue were diluted with sterile physiological solution in a dilution of 1:2. This dilution was added to the cell pellet so as to make up, together with the cells, the amount of material necessary to fill the tendon damage; said amount was previously calculated by means of ultrasound measurements. Lastly, the sample of BMSC cells added to the fibrinogen was aspirated into a syringe and injected into the tendon lesion by means of a needle, which is inserted into the lesion under ultrasound guidance. At the end of the injection, using the same needle, a small amount of thrombin was injected to achieve the clotting of the preparation. The area treated was disinfected and a bandage was applied.

In order to quantify the formation of fibroblastoid cells (expressed as ratio of the number of colonies obtained and number of plated cells) of a specific bone marrow sample, after having counted the nucleated cells, 100 microlitres of the sample were double plated in culture dishes in complete F12 (450 ml of medium, 50 ml of SFB (10%), 5 ml of glutamine 10×, 5 ml of streptomycin/penicillin, 5 ml of gentamycine). The plates were incubated at 37° C. in an atmosphere of 5% CO₂ for 14 days. The medium was changed twice a week and after 14 days it was eliminated, after which the colonies were coloured.

The horses treated underwent clinical and ultrasound tests every week for 8 weeks and after the 8 week period, they were tested each month. All the data gathered was compared using the ANOVA test. Significance was set at p<0.05. An average of 66.7 ml (±23.09 SD) of bone marrow was extracted. The average cell count before centrifuging with Ficoll was 635 CFU-f (±572.90 DS), while the average count of the CFU-f post-Ficoll was 1823.33 (±722.10 DS). The statistical comparison of these results was found to be statistically significant. The ultrasound tests that were carried out showed significance as early as three weeks from the treatment, both in terms of FPS and TLS values. These parameters remained significant in all the tests. At 8 weeks from the treatment, the subjects were discharged and given to their owners for a gradual training process. The use of the wing of the ilium as extraction site was found to be highly advantageous, both as far as the comfort and safety of the operator is concerned, and in relation to the amount of material extracted. Sedation with detomidine and butorphanol combined with a local anaesthetic proved to be sufficient in order to carry the procedure without having to resort to using general anaesthetic. Furthermore, having considerable quantities of bone marrow available also provides access to larger quantities of mononucleated cells that can be used fresh. As mentioned above, the use of fresh mononucleated cells implies considerable advantages in terms of the number of cells implanted than the results that can be achieved by using the bone marrow in toto. This is demonstrated by the pre and post-Ficoll counts of the CFU-F, carried out according to the present invention.

According to the invention, it is therefore possible to deliver considerable quantities of cells to the tendon lesion only, with the resultant advantage that the activation of the healing processes only takes place in the site of the injury, thus avoiding any complications involving inflammation affecting the whole tendon.

A further advantage according to the present advantage is the fact of using fresh cells, which do not require any in vitro expansion, enrichment or differentiation processes before they can be used. Indeed, procedures such as expansion, for example, necessarily require a significant increase in the costs of the procedure and, consequently, of the treatment as a whole. A further advantage lies in the fact that, whilst not carrying out any in vitro expansion, it is possible to directly inject into the lesion not all marrow extracted in toto, but only the stem cells that are useful for the repair of the lesion. This avoids having to inject all the components of the bone marrow that do not contribute in any way to repairing the injury, which may be a tendon injury, but may instead cause local inflammation.

A further advantage lies in the association of the fibrin glue/fibrinogen with the cell pellet, with the resulting aggregation of the stem cells which then, once they are directly injected into the lesion, remain only in the injection site and are not lost; in this way, an ideal substratum of development and replication is also guaranteed. The ultrasound guided injection of the cells into the lesion guarantees that the preparation is delivered precisely and safely into the lesion itself, thus providing an adequate number of cells only to the affected site; this eliminates the risk of the cells being lost. Furthermore, by using BMSC cells according to the present invention, in no case was an inflammatory reaction observed. From an ultrasonographic point of view, an initial recovery was already established three weeks from the treatment as far as the architecture of the collagen fibres is concerned; in addition to this was a reduction of the oedema inside the lesion, and repair that progressed over the subsequent tests. A gradual improvement of the FPS and TLS was also observed up to 12 weeks, when parameters were found that were comparable to those in healthy tendons. Observing strict resting later followed by gradual training proved to be extremely important for the purpose of returning to sporting activity.

The object of the present invention is therefore based on the capacity of the bone marrow mononuclear stromal cells to differentiate inside a specific microenvironment and produce support cells of the same kind as those present in the environment into which they are placed. For example, in this case, as they are placed for example in tendons, they will develop as tenocytes. As has already been pointed out, current techniques according to prior art which use bone marrow stromal cells to repair damage to tendons necessarily involve on the one hand, the selection of the mononucleated cells (BMSC) and their in vitro expansion, or their enrichment or even differentiation; and on the other, the use of the marrow exactly as it is, without selecting the cells themselves. In the first case, there is therefore the need to treat the selected stem cells in vitro, resulting in high costs which impact the costs of the treatment as a whole and require lengthy periods of time; in the second, there is the disadvantage that, by using the marrow as it is, a number of other useless components are also inserted into the lesion which may in fact be potentially damaging.

According to the present invention, said mononuclear stromal cells are preferably and advantageously autologous, with the advantage of avoiding any problems of rejection or compatibility between different subjects.

The use of bone marrow stromal cells, even ones that are not expanded, not enriched and not differentiated prior to injection into the lesion, enables the injury to heal by means of the regeneration of the tendinous tissue, thanks to the formation of tenocytes, thus avoiding the formation of scar tissue, which would result in permanent damage.

1. Fresh bone marrow mononuclear stromal cells (BMSC) for the treatment of injuries to tendons and/or ligaments in mammals.

2. Fresh bone marrow mononuclear stromal cells (BMSC) for the preparation of a medicament for the treatment of injuries to tendons and/or ligaments in mammals.

3. Fresh bone marrow mononuclear stromal cells (BMSC) according to claim 1, characterised in that they are autologous cells.

4. Cells according to claim 1, characterised in that said mammals are humans, horses, dogs, camels, sheep.

5. Cells according to claim 4, characterised in that said mammals are racehorses.

6. Cells according to claim 1, characterised in that they are treated with fibrin glue/fibrinogen.

7. Process for the treatment of injuries to tendons and/or ligaments in a mammal, that involves the use of fresh bone marrow mononuclear stromal cells (BMSC).

8. Process according to claim 7, characterised in that said cells are autologous.

9. Process according to claim 7, characterised in that said mammals are racehorses.

10. Process for the preparation of bone marrow mononuclear stromal cells (BMSC) that includes the following phases:
providing a sample of bone marrow
selecting the mononuclear stromal cells (BMSC) from said sample of bone marrow
treatment of said cells with fibrin glue/fibrinogen obtaining fresh bone marrow mononuclear stromal cells (BMSC).

11. Composition for the treatment of injuries to tendons and/or ligaments in mammals, in particular humans, horses, dogs, camels, sheep, and more specifically racehorses, which includes fresh bone marrow mononuclear stromal cells (BMSC).

12. Composition according to claim 11, characterised in that said cells are autologous.

13. Kit for the treatment of injuries to tendons and/or ligaments in mammals, in particular humans, horses, dogs, camels, sheep, and more specifically racehorses, which includes:
a syringe that is suitable for obtaining a bone marrow sample
a container with the addition of fibrin glue/fibrinogen
bone marrow mononuclear stromal cells (BMSC).

14. A method for the treatment of injuries to tendons and/or ligaments in a mammal, in particular human, horse, dog, camel, sheep, that includes administering to the mammal a composition of fresh bone marrow mononuclear stromal cells (BMSC).

15. Method according to claim 14, characterised in that said mammal is a racehorse.

16. Method according to claim 15, characterised in that said treatment is carried out by delivering said fresh cells (BMSC) directly to the site of the injury.

17. Method according to claim 15, characterised in that bone marrow is extracted from the wing of the ilium.

18. Method according to claim 17, characterised in that said cells are autologous.

19. Kit for the treatment of injuries to tendons and/or ligaments in mammals, in particular humans, horses, dogs, camels, sheep, and more specifically racehorses, which includes:
a composition of bone marrow mononuclear stromal cells (BMSC) in a suitable medium.
means for administering said composition to a mammal affected by an injury to tendons and/or ligaments.
means for determining the exact position where said composition should be administered in correspondence to the lesion.
